(19) United States
(12) Patent Application Publication
(10) Pub. No.: US 2010/0242964 A1
(43) Pub. Date: Sep. 30, 2010

(54) Device for providing heated air for an individual to breathe

(76) Inventor: Franklin C. Reynolds, South Windsor, CT (US)

Correspondence Address: ALIX YALE & RISTAS LLP, 750 MAIN STREET, SUITE 1400, HARTFORD, CT, 06103, US

(21) Appl. No.: 12/383,491

(22) Filed: Mar. 25, 2009

(51) Int. Cl. A61M 16/00

(52) U.S. Cl. 128/204.17

(57) ABSTRACT

A device for providing heated air for an individual to breathe. The device comprises a heating circuit with a heating element that is surrounded and supported by a pad. The pad is attachable to an article of clothing. The heating element and pad form a heating unit that is positionable proximate an individual's breathing region when the article of clothing is worn by the individual. The pad is configured to allow heat generated by the heating element to heat the air to be breathed in by the individual.

### BACKGROUND OF THE DISCLOSURE

The present disclosure relates to heating units, and more particularly, to a heating unit attachable to a scarf or like article of clothing for providing heated air for an individual to breathe in cold weather conditions.

Symptoms of some ailments such as bronchial diseases are often aggravated by breathing cold air. In fact, colder environments can pose severe health risks to many patients suffering from diseases such as chronic obstructive pulmonary disease, asthma, bronchitis, emphysema and angina pains, amongst others. Even in individuals who are free from such ailments, breathing in cold air can cause discomfort and health risks. Portable devices for warming an individual's body in cold environments are known, but such devices do not allow users to avoid breathing in cold air.

### SUMMARY

There is a need for a device that provides heated air for a user to breathe while in cold environments, and thus avoid discomfort or aggravation of symptoms and health risks. The present disclosure is directed to a heat generating device that is attachable to and preferably removable from a scarf or like clothing article and can be placed proximate a user's nose and mouth during use. Alternatively, the device can be provided as a combination with a scarf or like article.

According to one embodiment, a heating device is configured to heat air to be breathed in by an individual. The heating device comprises a heating circuit and a pad. The heating circuit has a heating element, a power source and a switch. The switch is configured to pass an electric current through the heating element from an electric power source when in the ON position. The pad generally surrounds and supports the heating element. The pad has an outer surface that is attachable to an article of clothing. The pad is configured to allow heat that is generated by the heating element when the switch is in the ON position to be breathed in by the individual in the form of hot air.

In another embodiment of the heating device, a heating circuit has a nichrome heating element connected to a power source by a length of double strand copper wire and a switch. The switch is configured to selectively provide electric communication between the heating element and the power source when in the ON position. A pad generally surrounds the heating element. The pad has an outer surface that is attachable to an article of clothing. At least one layer of adhesive separates and is attached to both the heating element and pad. The pad, tape and heating element form a heating unit that is positionable proximate the breathing region of an individual. The tape and pad are configured to allow heat generated by the heating element when the switch is in the ON position to heat air to be breathed in by the individual.

In yet another embodiment, an article of clothing is configured to provide heated air to the breathing region of an individual. The article of clothing comprises a unit of fabric over the individual's breathing region and a heating unit. The heating unit has a heating element that is configured to generate heat. A pad is attached to the unit of fabric and generally surrounds the heating element. The pad guards the heating element from physical contact with the individual. The heating unit is positionable proximate an individual's breathing region. The pad and adhesive material are configured to allow heat generated by the heating element to heat air to be breathed in by the individual.

### BRIEF DESCRIPTION OF THE DRAWING

Aspects of the preferred embodiment will be described in reference to the Drawing, where like numerals reflect like elements:

FIG. 1 shows an unpacked view in partial schematic of a heat generating device according to the disclosure; and

FIG. 2 shows the heat generating device of FIG. 1 engaged with a scarf.

### DETAILED DESCRIPTION OF THE PREFERRED EMBODIMENT

With reference to the drawing wherein like numerals represent like parts throughout the Figures, an embodiment of a heating unit for use with a scarf or like article is shown.

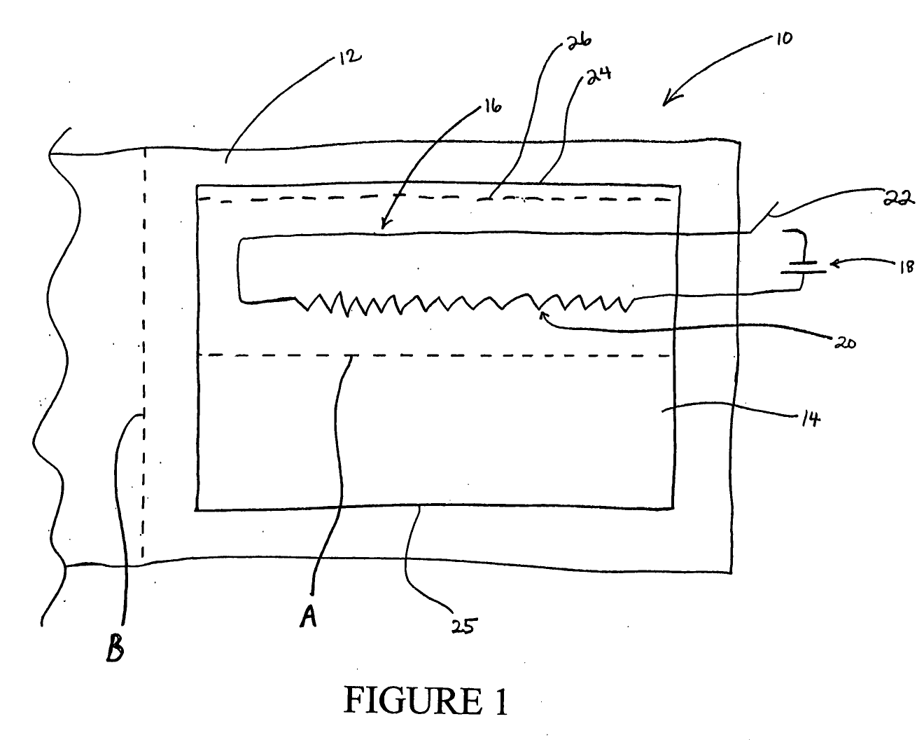

An embodiment of the disclosed heating device 10 is shown in FIG. 1 in unpacked form. The heating device 10 comprises a heating circuit 16 that is attached to a pad 12 via adhesive 14. The heating circuit 16 has a power source 18, heating element 20 and switch 22. In this embodiment, an adhesive strip 14 is positioned between the heating element 20 and the pad 12 and supports the heating element 20 within the pad 12. More particularly, the heating element 20 is enveloped by the adhesive strip 14 which is enveloped by the pad 12. The heating element-pad combination is referred to herein as the heating unit 11.

Preferably, the adhesive strip 14 is a piece of heat resistant tape or similar heat resistant material. The adhesive strip 14 is fixed along one edge 24 to the pad 12. In this embodiment, attachment is represented by stitch marks 26, but such form of attachment is not limiting. The heating circuit 16 is stuck to the adhesive strip 14 generally toward the attached edge 24. As seen in FIG. 1, as a safety enhancement measure, at least the entire heating element 20 is contained within the boundaries of the adhesive strip 14. Here, the adhesive strip 14 is then folded inward generally in half along line A so that the unattached edge 25 approximately meets the attached edge 24. This folding and meeting of the edges 24 and 25 contains the heating element 20 generally within a pocket formed by the adhesive strip 14. It should be noted that the approximate meeting of edges 24 and 25 in this embodiment is not limiting.

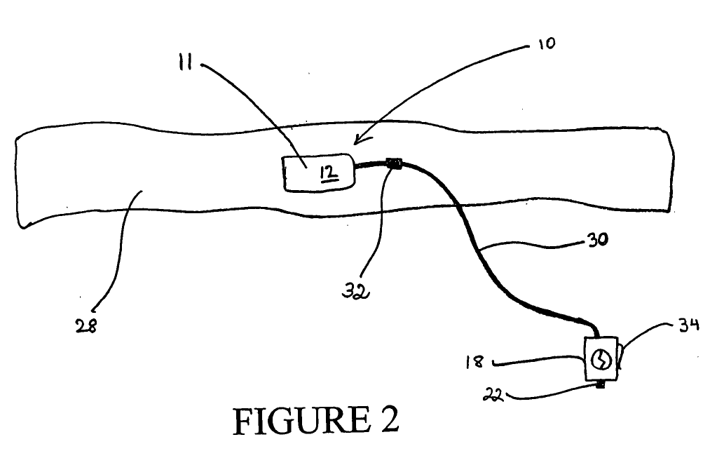

In this embodiment, the pad 12 is then folded inward generally along line B so that the entire heating element-adhesive strip assembly is generally enveloped by the pad 12. In some embodiments, the overlapping outer edges of the pad 12 can be joined to each other via stitching, adhesive or any other known method of attachment to form a heating unit 11. The heating device 10 can then be attached to a scarf 28 or like article (see FIG. 2). While this embodiment features a separate adhesive strip 14 and pad 12 that are attached, other embodiments exist wherein a single component possesses the properties of both the adhesive and pad. Further, attachment of the heating element 20 and pad 12 via adhesive 14 can be substituted with any known relatively heat resistant method of attachment.

As shown in FIG. 2, the wire 30 connecting the power source 18 with the heating element 20 (hidden beneath the pad 12 in FIG. 2) is fit with a disconnect coupling 32. Disconnection of the power source 18 and heating unit 11 allows for easy disinfection of the heating device 10 and/or replacement or recharging of batteries within the power source 18.

In this embodiment, the heating unit 11 and scarf 28 are fit with cooperating patches of hook and pile material such as Velcro® to assist in attachment of the heating device 10 to the scarf 28. The cooperating hook and pile patches in this embodiment can be substituted with another attachment means such as a hook assembly, pin, adhesive or the like. The means for attachment enable the heating device 10 to be removed and re-attached to the same or another scarf or article.

After connecting the heating device 10 to a scarf or like article, a typical user can wrap the scarf around himself so that the heating unit 11 is lined up proximate his nose and mouth (breathing region). The heating unit 11 is held in place proximate the breathing region via the pressure from the wrapped scarf and/or another piece of outerwear, like a jacket. Once the switch 22 is turned to the ON position, the power source 18 powers the heating element 22 which generates heat from the heating unit 11 to the user's breathing region. The user can attach the power source 18 to an article of clothing via a clip 34, thus freeing both hands. The powered heating element constantly delivers heated air for the user to breathe. The device thus enables the user to venture into cold environments and avoid the dangers or discomfort associated with breathing in cold air.

The heating device 10 can be easily removed from the scarf 28 as desired by the user. The removability enables the scarf to be washed periodically or worn separately. The heating unit portion 11 of the device 10 can also be periodically sanitized by spraying the outer surface of the pad 12 with disinfectant spray. When the heating device 10 is not in use, for example during summer months, the heating unit portion 11 can be separated from the wire 30 at the disconnect coupling 32 and stored in a sanitary environment like a refrigerator.

In a preferred embodiment, the heating element 20 is an approximately three to approximately four inch long piece of nichrome solid core wire. The adhesive strip 14 is a strip of heat resistant cloth tape fit to size. The outer edges of the pad 12 are sewn to create a “pocket” for the heating element and adhesive strip. An approximately twenty-five to approximately forty inch long double twist strand of twenty-two gauge copper wire provides an electric connection between the heating element 20 and power source 18. This particular embodiment employs a power source 18 that is a three volt two D-cell battery pack with an on/off switch. A clip 34 or like element protrudes from the outer surface of the battery pack 18a for attachment of the battery pack to a user's belt or another clothing article during use of the heating device.

This particular embodiment was tested and operated for over five hours, continuously providing substantial heat to the breathing region of an individual. The intensity of heat generated was substantial enough to deliver constant hot air to the individual to breathe while outdoors in an approximately 25° F. environment.

Additional embodiments exist wherein the pad 12 is configured to provide access to an individual's breathing region for a tube or tubes from a portable oxygen tank.

While a preferred embodiment has been set forth for purposes of illustration, the foregoing description should not be deemed a limitation of the invention herein. Accordingly, various modifications, adaptations and alternatives may occur to one skilled in the art without departing from the spirit of the invention and scope of the claimed coverage.

1. A heating device for providing heat to the breathing region of an individual, comprising:
a heating circuit with a heating element, a power source and a switch configured to selectively provide electric communication between the heating element and power source when in an ON position;
a pad supporting and generally surrounding the heating element arranged to protect an individual against direct physical contact with the heating element and having an outer surface that is attachable to an article of clothing; wherein
the pad is configured to be positioned proximate an individual's breathing region and allow heat generated by the heating element when the switch is in the ON position to heat the air to be breathed in by the individual.

2. The heating device of claim 1, wherein the outer surface of the pad and the article of clothing are attachable via cooperating hook and pile material.

3. The heating device of claim 1, wherein the heating circuit further comprises a strand-of insulated copper wire with a length of between about 30 and about 40 inches for providing electric communication between the heating element and the power source when the switch is in the ON position.

4. The heating device of claim 3, wherein the copper wire is fit with a disconnect coupling for physically separating the heating element from the power source.

5. The heating device of claim 1, wherein the power source is housed within a casing that is removably attachable to an article of clothing.

6. The heating device of claim 5, wherein the casing is removably attachable to an article of clothing via a clip on the outer surface of the casing.

7. The heating device of claim 1, wherein the article of clothing is a scarf with a receiving surface that is configured to be mate-able with and attachable to a surface on the pad.

8. The heating device of claim 7, wherein when the scarf is wrapped around the individual, the force of the wrapped scarf holds the pad relatively stationary proximate the individual's breathing region.

9. The heating device of claim 1, wherein the heating element is supported within the pad by an adhesive material.

10. The heating device of claim 9, wherein the adhesive material is at least one layer of heat resistant tape that is positioned between and attached to each of the heating element and the pad.

11. The heating device of claim 1, wherein the heating element is a resistance element with a length of about three to about four inches.

12. The heating unit of claim 1, wherein the heating element and pad form a heating unit that is configured and positioned to allow access to an individual's breathing region for at least one external tube.

13. A heating device for providing heat to the breathing region of an individual, comprising:
a heating circuit with a nichrome heating element connected to a power source by a length of double strand copper wire and a switch configured to effect electric communication between the heating element and power source when in an ON position;
a pad generally surrounding the heating element and having an outer surface that is attachable to an article of clothing;
at least one layer of heat resistant cloth tape positioned between and attached to both of the heating element and the pad and configured to support the heating element within the pad; wherein the pad, tape and heating element form a heating unit that is positionable proximate the breathing region of an individual and the tape and pad are configured to allow heat generated by the heating element when the switch is in the ON position to heat air to be breathed in by the individual.

14. The heating device of claim 13, wherein the nichrome heating element has a thickness of approximately 28 gauge and has a length of approximately 3.5 inches, each strand of the double strand copper wire has a thickness of approximately 22 gauge and the total length of the copper wire is approximately 33 inches, and the power source is a 2 D-cell battery pack.

15. The heating device of claim 14, wherein the copper wire is fit with a disconnect coupling that is positioned approximately 2 inches from the pad.

16. An article of clothing configured to provide heated air to the breathing region of an individual, comprising:
a unit of fabric over the individual's breathing region;
a heating unit having
a heating element configured to generate heat; and
a pad attached to the unit of fabric, generally surrounding the heating element and guarding the heating element from physical contact with the individual; wherein
the heating unit is positionable proximate an individual's breathing region and the pad is configured to allow heat generated by the heating element to heat air to be breathed in by the individual.

17. The article of clothing of claim 16, wherein the heating element is a resistance element that is electrically connected to a power source.

18. The article of clothing of claim 17, wherein the power source is a battery pack that is electrically connected to the heating element by a length of insulated copper wire.

19. The article of clothing of claim 18, wherein the power source is detachable from the heating element via a disconnect coupling positioned within the length of the copper wire.

20. The article of clothing of claim 16, wherein the heating unit is detachable from the unit of fabric.
